(19) United States
(12) Patent Application Publication
(10) Pub. No.: US 2005/0003268 A1
(43) Pub. Date: Jan. 6, 2005

(54) Battery housing configuration

(76) Inventors: Erik R. Scott, Maple Grove, MN (US); Carl D. Wahlstrand, Lino Lakes, MN (US); Robert M. Skime, Coon Rapids, MN (US); Craig L. Schmidt, Eagan, MN (US); James M. Haase, Maplewood, MN (US)

Correspondence Address: SHUMAKER & SIEFFERT, P. A., 8425 SEASONS PARKWAY, SUITE 105, ST. PAUL, MN, 55125, US

(21) Appl. No.: 10/835,245

(22) Filed: Apr. 29, 2004

Related U.S. Application Data
(60) Provisional application No. 60/471,262, filed on May 16, 2003.

(51) Int. Cl. H01M 2/02; H01M 2/30; A61N 1/02

(52) U.S. Cl. 429/176; 429/181; 429/7; 607/116

(57) ABSTRACT

The invention is directed to techniques for decreasing the volume and thickness of a hermetic battery that includes an electrode stack contained within a hermetic housing. In particular, the invention is directed to batteries that have a non-uniform thickness as defined by the hermetic housing. A battery according to the invention includes a battery housing with at least two battery housing portions that define different thicknesses. For example, a first portion of the battery housing may have first thickness and house the battery, while a second portion of the battery housing has a second thickness and includes one or more feedthroughs. The second thickness may be greater than the first thickness.

This application claims the benefit of U.S. provisional application Ser. No. 60/471,262, filed May 16, 2003, the entire content of which is incorporated herein by reference.

### TECHNICAL FIELD

The invention relates to batteries, such as batteries for implantable medical devices.

### BACKGROUND

Implantable medical devices (IMDs) may perform a variety of functions, including patient monitoring and therapy delivery. In general, it is desirable to design an IMD to be as small as possible, e.g., in terms of volume, footprint, and/or thickness, while still effectively performing its intended function. For example, decreasing the size of an IMD can increase the number of possible locations in which the IMD can be practically implanted. In addition, a smaller IMD can limit the extensiveness of surgery, reduce the likelihood of infection or rejection of the implant, and improve the comfort, and in some cases cosmetic appearance, of a patient after implantation. In other words, a smaller IMD may be more clinically acceptable than its larger counterparts.

Typically, an IMD includes a housing that contains substantially all of the components of the IMD, and defines the size and shape of the IMD. The size and shape of the IMD housing is, in turn, dependant on the sizes and shapes of the components within the IMD housing. In particular, large components common to most IMDs have a substantial impact on the overall size and shape of an IMD housing. Common large components for an IMD include a battery and a hybrid circuit that includes digital circuits, e.g., integrated circuit chips and/or a microprocessor, and analog circuit components.

Many types of batteries useful for powering an IMD can emit materials that would be harmful to the patient in which the IMD is implanted and to the other components of the IMD. Consequently, existing IMDs typically use hermetic batteries, e.g., batteries contained within a hermetically sealed housing or case, as a source of power. However, the need to make the housing or case of the battery hermetic limits the thinness and shapes that the hermetic battery may have, e.g., due to need for hermetic feedthroughs and the type of welding or brazing required to seal the pieces, e.g., halves, of a hermetic housing or case. In particular, existing efforts to reduce the size of IMD batteries have focused on reducing the thickness of entire IMD battery housings. However, the thickness reduction available as a result of such efforts is limited by the size of the feedthroughs necessary to maintain the hermeticity of the batteries.

### SUMMARY

In general, the invention is directed to techniques for decreasing the volume and thickness of a hermetic battery that includes an electrode stack within a hermetic housing. In particular, the invention is directed to batteries that have a non-uniform thickness as defined by the hermetic housing. A battery that includes a housing that defines a non-uniform thickness according to the invention may have a decreased volume and thickness relative to existing hermetic batteries that include housings that define a uniform thickness. Moreover, when a battery according to the invention is included within an implantable medical device (IMD), the size, e.g., volume, footprint, and/or thickness, of the IMD may be decreased relative to IMDs that include conventional hermetic batteries.

A battery according to the invention includes a battery housing with at least two portions that define different thicknesses. For example, a first portion of the battery housing has a first thickness and may house the electrode stack, while a second portion of the battery housing has a second thickness and includes one or more hermetic feedthroughs. Due to the size of the feedthroughs, the thickness of the second portion of the battery housing may be required to be greater than the thickness of the first portion of the battery housing. However, the overall volume and the thickness of a substantial portion of the battery is reduced by reducing the thickness of the first portion of the battery housing to the extent permitted by the size of the electrode stack therein. In other words, the thickness of the second portion of the battery housing may be defined by the size and shape of the feedthroughs, and the thickness of the first portion of the battery housing may be defined by the size and shape of the electrode stack therein, e.g., by the thickness of the electrode stack. As used herein, the “thickness” of a battery housing refers to the smallest of its three dimensions, i.e., length, width and thickness.

In some embodiments, a battery according to the invention may be a module of a modular IMD that includes at least one other module. By distributing components of an IMD amongst modules rather than including them within a single, rigid housing, the IMD may be shaped and configured for implantation at locations within patient for which implantation of conventional IMDs is deemed undesirable. To further increase the versatility of a modular IMD, the modules may be at least partially encapsulated by a member that generally provides a smooth interface between the modules and body tissue. Alternatively, a battery according to the invention may be part of a non-modular IMD, in which substantially all the components the IMD are located within a single housing.

In one embodiment, the invention is directed to a battery comprising an electrode stack, a feedthrough coupled to the electrode stack, and a battery housing including a first portion that houses the electrode stack and a second portion that includes the feedthrough, wherein a thickness of the second portion is greater than a thickness of the first portion.

In another embodiment, the invention is directed to a battery comprising an electrode stack, a feedthrough coupled to the electrode stack, and a battery housing that houses the electrode stack and includes the feedthrough, wherein the battery housing includes a first portion with a thickness defined by the electrode stack, and a second portion with a thickness defined by the feedthrough.

In another embodiment, the invention is directed to an implantable medical device comprising a housing and a battery located within the housing. The battery comprises an electrode stack to provide power for the implantable medical device, a feedthrough coupled to the electrode stack, and a battery housing including a first portion that houses the electrode stack and a second portion that includes the feedthrough, wherein a thickness of the second portion is greater than a thickness of the first portion.

In another embodiment, the invention is directed to a modular implantable medical device comprising a plurality of interconnected modules, wherein one of the modules comprises a battery. The battery comprises an electrode stack to provide power for the modular implantable medical device, a feedthrough coupled to the electrode stack, and a battery housing including a first portion that houses the electrode stack and a second portion that includes the feedthrough, wherein a thickness of the second portion is greater than a thickness of the first portion.

In another embodiment, the invention is directed to a method of making a battery that comprises an electrode stack, a feedthrough coupled to the electrode stack, and a battery housing. The method comprises forming at least one of a plurality of pieces of the housing such that a thickness of a first portion of the battery housing is less than a thickness of a second portion of the battery housing. The method further comprises positioning the electrode stack within the first portion of the battery housing, and positioning the feedthrough to pass through the battery housing at the second portion.

In another embodiment, the invention takes the form of a battery that includes an electrode stack, a fill port, and a battery housing that houses the electrode stack and includes the fill port. The battery housing includes a first portion with a thickness defined by the electrode stack, and a second portion with a thickness defined by the fill port.

The invention may be capable of providing one or more advantages. For example, reduction of volume and/or thickness of a battery of a modular or non-modular IMD may allow the volume and/or thickness of the IMD to decrease. Decreasing the size of the IMD in this manner can increase the number of possible locations in which the IMD can be practically implanted. In addition, a smaller IMD can limit the extensiveness of surgery, reduce the likelihood of infection, and improve the comfort and cosmetic appearance of a patient after implantation. In some embodiments, a thin battery according to the invention may facilitate reduced thickness of a modular IMD for cranial implantation. A thinner modular IMD may be more clinically acceptable for cranial implantation due to, for example, the reduced likelihood of skin erosion on the scalp above the IMD.

Further, in some embodiments, a battery according to the invention may include space for a component to fit over the first portion of the battery housing, which has a thickness that is less than that of the second portion of the battery housing. In a modular IMD embodiment, the component may be another module of the IMD. In either case, stacking a module or other components of an IMD on top of the battery housing may decrease another aspect of the size of the IMD, i.e., the footprint of the IMD.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a conceptual diagram illustrating an example implantable medical device (IMD) that includes a hermetic battery according to the invention implanted on the cranium of a patient.

FIG. 2 is a top-view diagram further illustrating the IMD of FIG. 1 implanted on the cranium of the patient.

FIG. 3 is a top-view diagram further illustrating the IMD of FIGS. 1 and 2.

FIGS. 4A and 4B are side-view diagrams of example batteries that include battery housings with non-uniform thicknesses according to the invention.

FIG. 5A is a top-view diagram of the battery of FIG. 4A.

FIG. 5B is a top-view diagram of another example battery with a non-uniform thickness according to the invention.

FIG. 6 is a perspective diagram illustrating an example battery with a non-uniform thickness that is curved along at least one axis.

FIGS. 7A and 7B are side-view diagrams illustrating the example batteries of FIGS. 4A and 4B in conjunction with additional components of an IMD.

FIG. 8 is an exploded perspective view illustrating the battery of FIG. 4A.

FIGS. 9A-9C are exploded top, side, and perspective views, respectively, that illustrate another example battery.

FIG. 10 is a flow diagram illustrating an example method of manufacture for a battery according to the invention.

FIG. 11 is a side view illustrating another example IMD that includes a battery with a non-uniform thickness according to the invention.

### DETAILED DESCRIPTION

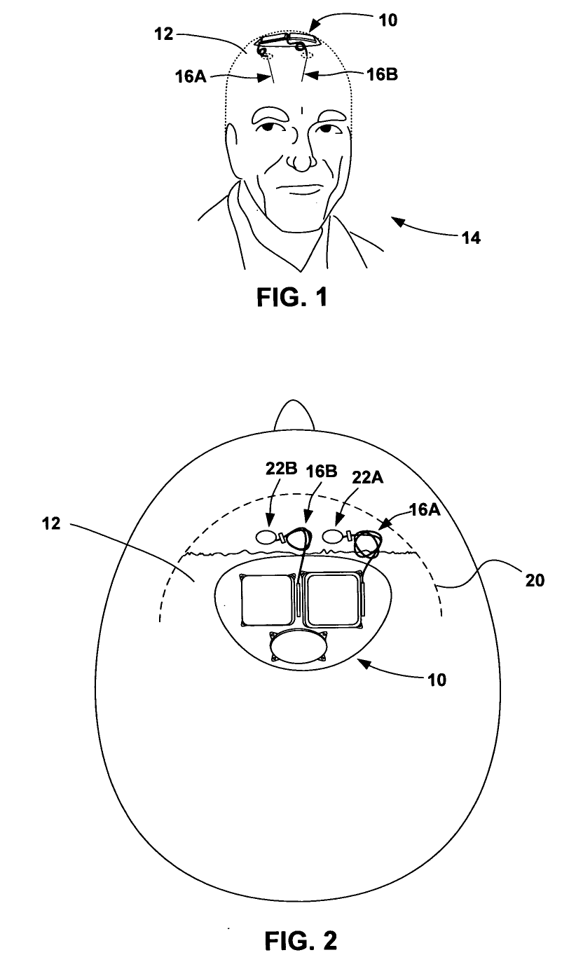

FIG. 1 is a conceptual diagram illustrating an example implantable medical device (IMD) 10 that includes a hermetic battery according to the invention. As will be described in greater detail below, the battery includes an electrode stack within a hermetic housing that defines a non-uniform thickness. Such a battery may have a decreased volume and thickness relative to existing hermetic batteries that include housings that define a substantially uniform thickness. Moreover, the size, volume, footprint, and/or thickness of IMD 10 may be decreased relative to conventional IMDs due to the use of a battery according to the invention.

A battery according to the invention includes a battery housing with at least two portions that define different thicknesses. For example, a first portion of the battery housing may have a first thickness for housing an electrode stack. A second portion of the battery housing may have a second thickness and may include one or more hermetic feedthroughs. Due to the size of the feedthroughs, the thickness of the second portion of battery housing may be required to be greater than the thickness of the first portion of the battery housing. In addition, the overall volume and the thickness of a substantial portion of the battery may be reduced by reducing the thickness of the first portion of the battery housing to the extent permitted by the size of the electrode stack therein. In other words, the thickness of the first portion of the battery housing may be defined by the size and shape of the electrode stack therein, e.g., the thickness of the electrode stack, and the thickness of the second portion of the battery housing may be defined by the size and shape of the feedthroughs. As used herein, the “thickness” of a battery housing refers to the smallest of its three dimensions, i.e., length, width and thickness.

In the embodiments illustrated in FIGS. 1-3, IMD 10 takes the form of a cranially implantable modular IMD that delivers neurostimulation to a patient 14. Modular IMD 10 includes a plurality of separately housed and flexibly interconnected modules that include the various components of IMD 10, and one of the modules includes a battery according to the invention. In other embodiments, a non-modular IMD in which substantially all of the IMD components are located in a single device housing may include a battery according to the invention within the housing.

In both types of IMDs, it may be beneficial to reduce the size of components, such as a battery, in order to reduce the overall size of the IMD. Further, the invention is not limited to embodiments in which a modular or non-modular IMD is a neurostimulator, or to cranially implanted IMDs. In other words, any type of IMD, such as an implantable neurostimulator, implantable pump, pacemaker, implantable cardioverter-defibrillator, implantable monitor, or the like, configured for implantation anywhere in a human or animal body, may include a battery according to the invention.

In the embodiment illustrated in FIG. 1, modular IMD 10 is implanted on the cranium 12 of patient 14, and comprises a plurality of separately housed and flexibly interconnected modules. By distributing components of IMD 10 amongst modules rather than including them within a single, rigid housing, IMD 10 may be shaped and configured for implantation at locations within patient 14 for which implantation of conventional IMDs is deemed undesirable. Further, the flexibility of the interconnection between modules of IMD 10 may allow multiples degrees of freedom of movement between the modules, which in turn may allow the implantable medical device to conform to such areas, and in particular embodiments, to conform to surfaces within patient 14 such as the surface of cranium 12.

In the illustrated example, IMD 10 is coupled to two leads 16A and 16B (collectively “leads 16”) that extend through holes within cranium 12, and into the brain of patient 14. In exemplary embodiments, each of leads 16 carries a plurality of electrodes, and IMD 10 delivers stimulation to the brain of patient 14 via the electrodes. Modular IMD 10 may be coupled to any number of leads 16, and in some embodiments is not coupled to any leads 16. In some embodiments, for example, IMD 10 may carry integrated electrodes.

Because IMD 10 can be implanted on cranium 12 of patient 14 rather than more remotely from the brain of patient 14, such as within an subclavicular region of patient 14, the problems associated with the use of long leads needed to allow a remotely implanted IMDs to access the brain may be diminished or avoided. These problems include the requirement of tunneling under the scalp and the skin of the neck, increased surgery and recovery time, an additional procedure under general anesthesia, risk of infection or skin erosion along the track through which the leads are tunneled, and risk of lead fracture due to torsional and other forces caused by normal head and neck movements.

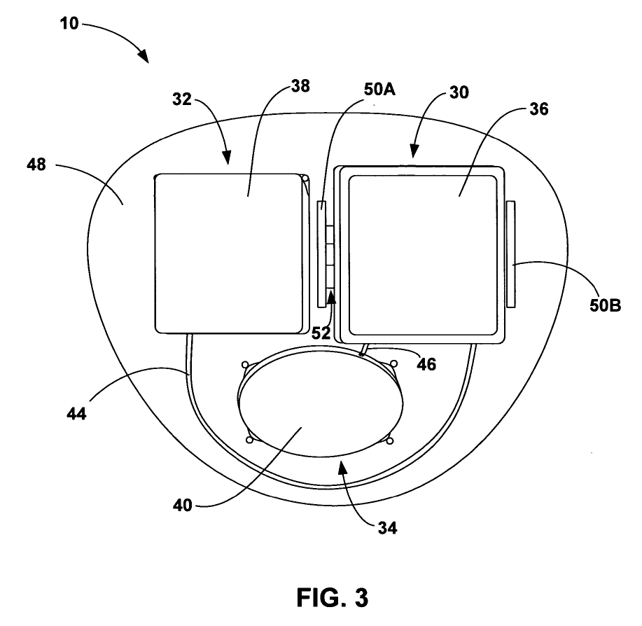

FIG. 2 is a top-view diagram further illustrating IMD 10 implanted on cranium 12 of the patient 14. In order to implant IMD 10 on cranium 12, an incision 20 is made through the scalp of patient 14, and a resulting flap of skin is pulled back to expose the desired area of cranium 12. The incision may, as shown in FIG. 2, be generally shaped like a “C.” Such an incision is commonly referred to as a “C-flap” incision.

Holes 22A and 22B (collectively “holes 22”) are drilled through cranium 12, and leads 16 are inserted through holes 22 and into the brain of patient 14. Caps may be placed over holes 22 as is known in the art. Leads 16 are connected to IMD 10, either directly or via a lead extension, and IMD 10 is placed at least partially within a pocket formed using a hand or a tool beneath the scalp behind holes 22.

Once positioned as desired on cranium 12 within the pocket, IMD 10 may then be fixed to cranium 12 using an attachment mechanism such as bone screws. The skin flap may be closed over IMD 10, and the incision may be stapled or sutured. The location on cranium 12 at which IMD 10 is illustrated as implanted in FIG. 2 is merely exemplary, and IMD 10 can be implanted anywhere on the surface of cranium 12.

Because of the flexibility that may be provided by interconnect members of IMD 10 and/or a member of modular IMD 10 that at least partially encapsulates the modules of IMD 10 and may provide a smooth interface between the modules and body tissue, the IMD may be manipulated during implantation such that it conforms to cranium 12. For example, in some embodiments a surgeon can manipulate modular IMD 10 into conformance with cranium 12 while IMD 10 is on cranium 12 and fix modular IMD 10 into place using bone screws or the like. In other embodiments, the clinician may manipulate modular IMD 10 into conformance with cranium 12 with IMD 10 on and/or off of cranium 12, and IMD 10 may substantially retain the form into which it is manipulated. Further details regarding exemplary techniques for implanting IMD 10 on the cranium may be found in a commonly-assigned U.S. patent application Ser. No. 10/731,868 entitled “IMPLANTATION OF LOW-PROFILE IMPLANTABLE MEDICAL DEVICE,” filed Dec. 9,2003.

Because of the reduction in size of IMD 10 provided by use of a battery according the invention, IMD 10 may be more easily implanted. More specifically, decreasing the size of IMD 10 can increase the number of possible locations in which the IMD can be practically implanted. In addition, a smaller IMD can limit the extensiveness of surgery, reduce the likelihood of infection, and improve the comfort and cosmetic appearance of a patient after implantation. Further, a thinner modular IMD 10 may be more clinically acceptable for cranial implantation due to, for example, the reduced likelihood of skin erosion on the scalp above the IMD.

As mentioned above, IMD 10 may deliver stimulation to the brain of patient 14 to, for example, provide deep brain stimulation (DBS) therapy, or to stimulate the cortex of the brain. Cortical stimulation may involve stimulation of the motor cortex. IMD 10 may be used to treat any nervous system disorder including, but not limited to, epilepsy, pain, psychological disorders including mood and anxiety disorders, movement disorders (MVD), such as, but not limited to, essential tremor, Parkinson's disease, and neurodegenerative disorders.

However, IMD 10 is not limited to implantation on cranium 12. Indeed, IMD 10 may be implanted anywhere within patient 14. For example, IMD 10 can be implanted within the neck of patient 14, and deliver stimulation to the vagus nerve or the cervical region of the spinal cord.

IMD 10 may alternatively be implanted within a pectoral region or the abdomen of patient 14 to act as a diaphragmatic pacer, or to provide any of the monitoring and therapy delivery functions known in the art to be associated with cardiac pacemakers. Further, IMD 10 may be implanted in the upper buttock region and deliver spinal cord, urological or gastrological stimulation therapy, or may be configured to be implanted within the periphery, e.g., limbs, of patient 14 for delivery of stimulation to the muscles and/or peripheral nervous system of patient 14.

IMD 10 is not limited to embodiments that deliver stimulation. For example, in some embodiments IMD 10 may additionally or alternatively monitor one or more physiological parameters and/or the activity of patient 14, and may include sensors for these purposes. Where a therapy is delivered, IMD 10 may operate in an open loop mode (also referred to as non-responsive operation), or in a closed loop mode (also referred to as responsive). IMD 10 may also provide warnings based on the monitoring.

Further, in some embodiments IMD 10 can additionally or alternatively deliver a therapeutic agent to patient 14, such as a pharmaceutical, biological, or genetic agent. IMD 10 may be coupled to a catheter, and may include a pump to deliver the therapeutic agent via the catheter.

FIG. 3 is a top-view diagram further illustrating IMD 10. In the illustrated embodiment, IMD 10 includes three modules: a control module 30, a battery 32, and a recharge module 34. As shown in FIG. 3, modules 30, 32 and 34 include separate housings 36, 38 and 40, respectively. Modules 30, 32, and 34 may be interconnected via interconnect members 44 and 46. Details regarding the configuration and/or construction of interconnect members 44 and 46 to provide flexibility may be found in a commonly-assigned U.S. patent application Ser. No. 10/731,699, entitled “COUPLING MODULE OF MODULAR IMPLANTABLE MEDICAL DEVICE,” filed Dec. 9, 2003.

Battery 32 includes an electrode stack (not shown in FIG. 3) within a hermetic housing 38, i.e. battery housing 38. The electrode stack provides power for components of other modules, such as the control electronics within control module 30. Battery 32 may include any of a variety of types of electrode stacks, i.e., energy storage elements, known if the art.

The electrode stack of a battery 32 according to the invention typically includes positive electrode active material, negative electrode active material, and an electrolyte. The electrode stack may also include inert parts of the electrode material, such as binder materials and conductivity enhancers, e.g. carbon. In addition, the electrode stack may include a separator and one or more current collectors.

Typical configurations of the electrode stack include a coil configuration, a flattened coil or “jelly-roll” configuration, a flat plate configuration, a serpentine electrode configuration, and a ‘z’-folded electrode. Further, battery 32 may have any of a variety of known battery chemistries. For example, in embodiments, in which battery 32 is rechargeable, battery 32 may have a Lithium Ion, Nickel-Metal Hydride, or Nickel-Cadmium chemistry. The electrode stack may be configured, e.g., may have a thin wound coil construction, or a stacked or z-shaped non-coiled construction, to more easily fit within first portion of battery housing 38 which may be less than 5 millimeters thick, as will be described in greater detail below. Battery housing 38 may be hermetic, and may be formed of, for example, titanium, stainless steel, a ceramic, an alloy of aluminum or titanium, or a polymer metal laminate. Battery 32 may include an insulator within battery housing 38 to isolate battery housing 38 from the electrode stack.

As mentioned above, battery housing 38 defines a non-uniform thickness. The non-uniform thickness of battery housing 38 may lead to a reduced overall volume and a reduced thickness of at least a portion of battery 32 relative to conventional batteries with substantially uniform battery housing thicknesses. In the illustrated embodiment, the reduced thickness of battery 32 may, in turn, lead to a reduced thickness of IMD 10 relative to modular IMDs that include conventional batteries. For example, in accordance with an embodiment of the invention, a first portion of battery housing 38 has a first thickness for housing the electrode stack, while a second portion of battery housing 38 includes a second thickness and includes one or more hermetic feedthroughs (not shown in FIG. 3).

A feedthrough may connect an electrode of the electrode stack to conductors within interconnect member 44, which are in turn coupled to other components of IMD 10, such as a circuit board located within control module 30. Due to the size of feedthroughs, the thickness of the second portion of battery housing 38 may be required to be greater than the thickness of the first portion of battery housing 38. However, the overall volume and the thickness of a substantial portion of the battery 32 may be reduced by reducing the thickness of the first portion of battery housing 38 to the extent permitted by the size of the electrode stack therein. In other words, the thickness of the first portion of battery housing 38 may be defined by the size and shape of the electrode stack therein, and the thickness of the second portion of battery housing 38 may be defined by the size and shape of the one or more feedthroughs.

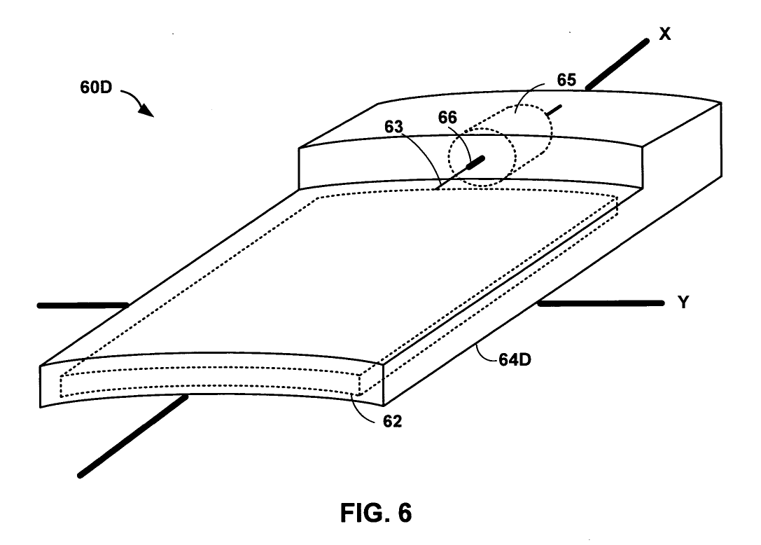

Battery housing 38 may have any shape, including the rectangular shape with rounded edges, i.e., the prismatic shape, illustrated in FIG. 3. Further, one or more surfaces of battery housing 38 may be curved along at least one axis, and preferably two axes. A battery including a housing that is curved along one axis is illustrated in FIG. 6. Further details regarding curvature of housings may be found in a commonly-assigned U.S. patent application Ser. No. 10/731,867 entitled “CONCAVITY OF AN IMPLANTABLE MEDICAL DEVICE,” filed Dec. 9,2003.

If the battery 32 is rechargeable, IMD 10 may include recharge module 34. A recharge coil, i.e., a secondary coil, within recharge module 34 inductively receives energy from an external recharging unit (not illustrated) that includes a primary coil through the skin of a patient to recharge the battery 32. Housing 40 need not be hermetic, and may be formed of materials such as silicone, polymers and ceramics. In the illustrated embodiment, the control electronics of control module 30 regulates the recharging and discharging of battery 32. Consequently, as shown in FIG. 1, recharge module 34 is coupled to control module 30 by an interconnect member 46 that encloses one or more conductors that allow transmission of energy inductively received by a coil to control module 30.

Control module 30 includes control electronics within housing 36, e.g., electronics that control the monitoring and/or therapy delivery functions of modular IMD 10, such as a microprocessor. Control module 30 may also include circuits for telemetry communication with external programmers or other devices within the housing. Housing 36 of control module 30 may be hermetic in order to protect the control electronics therein, and in exemplary embodiments is formed of a rigid material, such as titanium, stainless steel, or a ceramic.

In the illustrated embodiment, IMD 10 also includes lead connector modules 50A and 50B (collectively “lead connector modules 50”) formed within IMD 10 to receive leads or lead extensions coupled to leads. Conductors 52 extend from lead connector modules 50 to hermetic feedthroughs (not illustrated) within housing 36 of control module 30.

Modules 30, 32, and 34 can be configured in a variety of ways other than the exemplary configuration illustrated in FIG. 1. Additional exemplary groups of modules and configurations of modules are described in a commonly-assigned U.S. patent application Ser. No. 10/731,869 entitled “MODULAR IMPLANTABLE MEDICAL DEVICE,” filed Dec. 9, 2003. For example, modular IMD 10 can include additional batteries, modules that include additional memory that is accessible by the control electronics within control module 30, modules that include reservoirs for storing therapeutic agents and pumps for delivering therapeutic agents to patient 14, and modules that include sensors sensing physiological parameters, such as pressures or blood flows, or the activity level of patient 32.

In the illustrated embodiment, modules 30, 32 and 34 are coupled to a member 36, which may be made of a soft, biocompatible material. Member 48 at least partially encapsulates one or more housings of modules 30, 32, 34, and generally serves to provide a smooth interface between the modules and the body tissue. Member 48 may integrate modules 30, 32 and 34 into a desired form factor, but, where flexible, allow relative intermodule motion. In some embodiments, member 48 incorporates mechanical features to restrict intermodule motion to certain directions or within certain ranges. Member 48 may be made from silicone, and is some embodiments may be made from two or more materials of differing flexibility, such as silicone and a polyurethane. An exemplary polyurethane for this purpose is Tecothane®, which is commercially available from Hermedics Polymer Products, Wilmington, Mass. Member 36 may also be referred to as an “overmold,” but use of the term “overmold” herein is not intended to limit the invention to embodiments in which member 36 is a molded structure. Member 36 may be a molded structure, or may be a structure formed by any process.

Member 48 can be shaped to contour to cranium 12, e.g., may be curved along at least one axis, and may be contoured at its edges to prevent skin erosion on the scalp of patient 30. The flexibility and shape of member 48 may improve the comfort and cosmetic appearance of modular IMD 10 under the scalp. Further details regarding member 48, the curvature of the member, and techniques for restricting intermodular motion in a modular IMD 10 may be found in a commonly-assigned U.S. patent application Ser. No. 10/730,873 entitled “OVERMOLD FOR A MODULAR IMPLANTABLE MEDICAL DEVICE,” filed Dec. 9, 2003, and a commonly-assigned U.S. patent application Ser. No. 10/731,881 entitled “REDUCING RELATIVE INTERMODULE MOTION IN A MODULAR IMPLANTABLE MEDICAL DEVICE,” filed Dec. 9,2003.

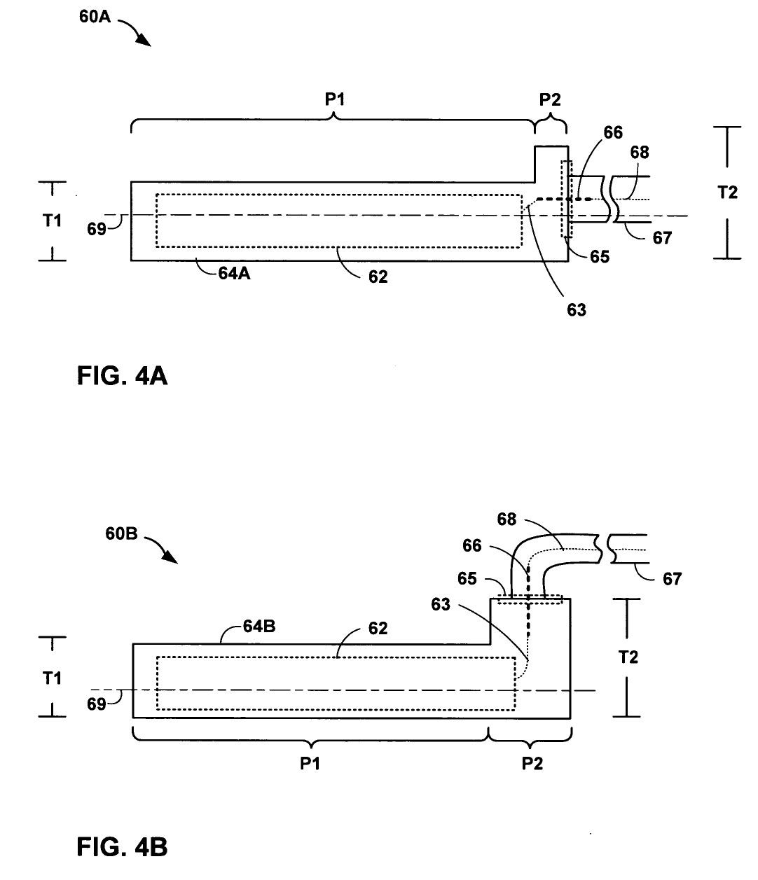

FIGS. 4A and 4B are side-view diagrams of exemplary batteries 60A, 60B that include battery housings 64 that define non-uniform thicknesses. Battery housings 64A, 64B with at least two thicknesses allow for a reduction in the overall volumes and thicknesses of a substantial portion of batteries 60A, 60B, as described above. As described above with reference to battery housing 38, housings 64A, 64B may have a generally prismatic shape, and may be formed of any of a variety of materials, as described above with reference to battery housing 38 of FIG. 3. Further, housings 64A, 64B house an electrode stack 62, which may include any of the components or configurations described above with reference to battery 32 of FIG. 3. Batteries 60A, 60B may have any of a variety of known battery chemistries, as described above with reference to battery 32 of FIG. 3.

In the illustrated embodiments, a first portion P1 of battery housings 64A, 64B has a first thickness T1 and houses electrode stack 62, while a second portion P2 of battery housings 64A, 64B has a second thickness T2 and includes at least one hermetic feedthrough 65. Feedthrough 65 may be connected to an electrode of electrode stack 62 by an interconnect 63, and may connect the electrode to other components of IMD 10, such as a circuit board within control module 30 (FIG. 3), via a feedthrough conductive element 66 and a conductive element 68 within an interconnect member 67. Housings 64A, 64B may include any number of feedthroughs 65 and feedthrough conductive elements 66, but will typically include a single feedthrough 65 with a respective feedthrough conductive element 66 coupled to one of the anode and cathode of electrode stack 62. In such embodiments, the electrode of electrode stack 62 that is not connected to feedthrough 65 may be connected to a portion of the battery housing 64A, 64B by another interconnect (not shown), which may be coupled to another conductive member (not shown) within interconnect member 67. Interconnect member 67 may correspond to interconnect member 44 illustrated in FIG. 3.

As illustrated in FIGS. 4A and 4B, feedthrough 65 includes, and conductive element 66 passes through, an insulative member. The insulative member may be formed of, for example, ceramic or a glass such as such as Cabal-12. Conductive element 66 may take the form of a conductive pin or rod, which may be formed of, for example, niobium, Ti-6V-4Al, aluminum, or molybdenum. The insulative member is hermetically sealed to conductive element 66 and the housing 64A, 64B by, for example, melting and bonding in the case of a glass insulative member, or brazing in the case of a ceramic insulative member. Although not illustrated in FIGS. 4A and 4B, housings 64A, 64B may be formed of two or more pieces that are hermetically sealed by welding.

Due to the size of feedthrough 65 necessary for hermaticity, the thickness T2 of the second portion P2 of battery housing 64A, 64B may be required to be greater than the thickness T1 of the first portion P1. In addition, the volume and the thickness of a substantial portion of batteries 60A, 60B may be reduced by reducing the thickness T1 of the first portion P1 of battery housings 64A, 64B to the extent permitted by the size of electrode stack 62 therein. In other words, the thickness of the first portion P1 of battery housing 64A, 64B may be defined by the size and shape of electrode stack 62 therein, and the thickness T2 of the second portion P2 of battery housing 64A, 64B may be defined by the size and shape of feedthrough 65.

In some embodiments, the thickness of the first portion P1 is within a range from approximately 1 mm to approximately 5 mm, and the thickness of the second portion is within a second range from approximately 3 mm to approximately 10 mm. In one exemplary embodiment, the thickness of the first portion P2 is approximately 3 mm, and the thickness of the second portion is approximately 6 mm. In some embodiments, the thickness T1 of the first portion P1 is less than approximately 80 percent of the thickness T2 of the second portion P2. In one exemplary embodiment, the thickness T1 of the first portion P1 is approximately 60 percent of the thickness T2 of the second portion P2.

As illustrated by FIGS. 4A and 4B, feedthrough 65 may be integrated into battery housings 64A, 64B in various manners. For example, as shown in FIG. 4A, feedthrough 65 may be positioned such that conductive element 66 may be coupled to a conductive element 68 carried by an interconnect member 67 extending from the end of second portion P2. In particular, conductive element 66 may extend out of battery housing 64A substantially parallel with a long axis 69 of battery 60A, as shown in FIG. 4A.

Alternatively, feedthrough 65 may be positioned such that conductive element 66 may be coupled to a conductive element 68 carried by an interconnect member 67 extending from the top of second portion P2, as shown in FIG. 4B. In particular, conductive element 66 may extend out of battery housing 64B in a manner such that the conductive element 66 is substantially perpendicular to long axis 69. The examples shown in FIGS. 4A and 4B are merely exemplary, and alternative embodiments could include different configurations of one or more feedthroughs 65 and interconnect members 67.

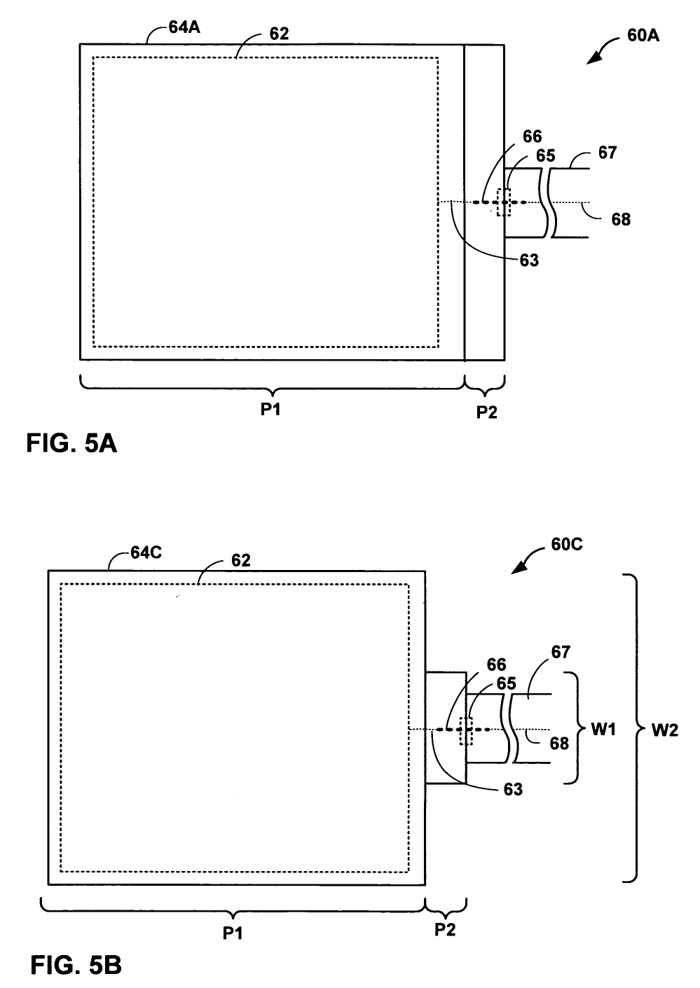

FIGS. 5A is a top-view diagram illustrating battery 60A of FIG. 4A, and FIG. 5B is a top-view diagram illustrating another example battery 60C that includes a battery housing 64C that defines a non-uniform thickness. In accordance with an embodiment of the invention, a first portion P1 of battery housings 64A, 64C has a first thickness for housing an electrode stack 62, while a second portion P2 of battery housings 64A, 64C has a second thickness and includes hermetic feedthrough 65. Feedthrough 65 may connect to an electrode of electrode stack 62 via an interconnect 63, and to other components of IMD 10 via feedthrough conductive element 66 and conductive element 68 within interconnect member 67.

In the example battery housing 64A shown in FIG. 5A, the entire width W2 of portion P2 has a thickness greater than the thickness of P1. Alternatively, in some embodiments, only width W1 of portion P2, which includes feedthrough 65, has a thickness greater than the thickness of P1. For example, in some embodiments, the part of portion P2 outside width W1 may define the same thickness as portion P1.

In another embodiment illustrated in FIG. 5B, battery housing 64C does not include the part of portion P2 outside width W1. In other words, the first portion P1 has a width W2, while the second portion P2 has a width W1, which is less than W2. A battery housing 64C configured in this manner may have a smaller volume than battery 64A, which may lead to a further reduction in the size of an IMD in which a battery 60C is included.

FIG. 6 is a perspective diagram illustrating an example battery 60D that has a battery housing 64D that is curved along one axis, labeled Y in the Figure, and defines two thicknesses. Any one or more surfaces of a battery housing may be curved, such as the top and bottom surfaces as illustrated by battery housing 64D in FIG. 6. As illustrated in FIG. 6, electrode stack 62 may also be curved to conform to the curvature of housing 64D. In some embodiments, batteries according to the invention may be curved along two axes, i.e., the axes labeled X and Y in FIG. 6.

Curvature along one or more axes may allow battery 60D to provide improved comfort and cosmetic appearance of a patient after implantation, e.g., to better conform to the cranium of the patient, to prevent clinical complications, and to reduce scalp erosion. For example, battery 60D may define an arc with a diameter that is similar to that of a typical cranium, e.g., approximately 5.75 inches. As indicated above, further details regarding the curvature of housings of a modular IMD may be found in a commonly-assigned U.S. patent application Ser. No. 10/731,867 entitled “CONCAVITY OF AN IMPLANTABLE MEDICAL DEVICE,” filed Dec. 9, 2003.

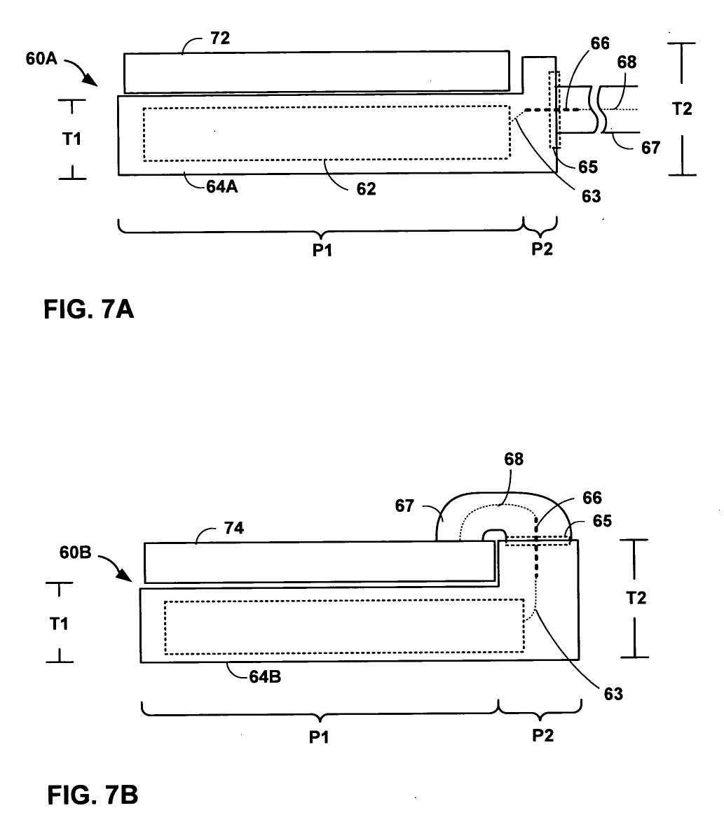

FIGS. 7A and 7B are side-view diagrams illustrating batteries 60A and 60B of FIGS. 4A and 4B in conjunction with additional components of an IMD. In particular, an IMD component, such as another module in modular IMD embodiments, may substantially fit within a space created in portion P1 when the thickness T1 of portion P1 is decreased to a thickness less than thickness T2. The component may include any component or module sized to substantially fit within the space created in portion P1. By stacking a component or module on battery housing 64, the footprint of an IMD may be decreased.

As shown in FIG. 7A, a component such as a recharging coil 72, which may correspond to recharge module 34 illustrated in FIG. 3, may fit on battery housing 64A within portion P1. As another example, FIG. 7B illustrates control electronics 74, which may correspond to control module 30 illustrated in FIG. 3, fitting on battery housing 64B. Control electronics 74 may be connected to electrode stack 62 via feedthrough conducting element 66 and conducting element 68 within interconnect 67. In modular embodiments, control electronics may take the form of a separately housed control module 30 that includes a circuit board carrying digital circuits, integrated circuit chips, a microprocessor, and/or analog circuit components. In non-modular embodiments, control electronics 74 may include the circuit board without a control module housing.

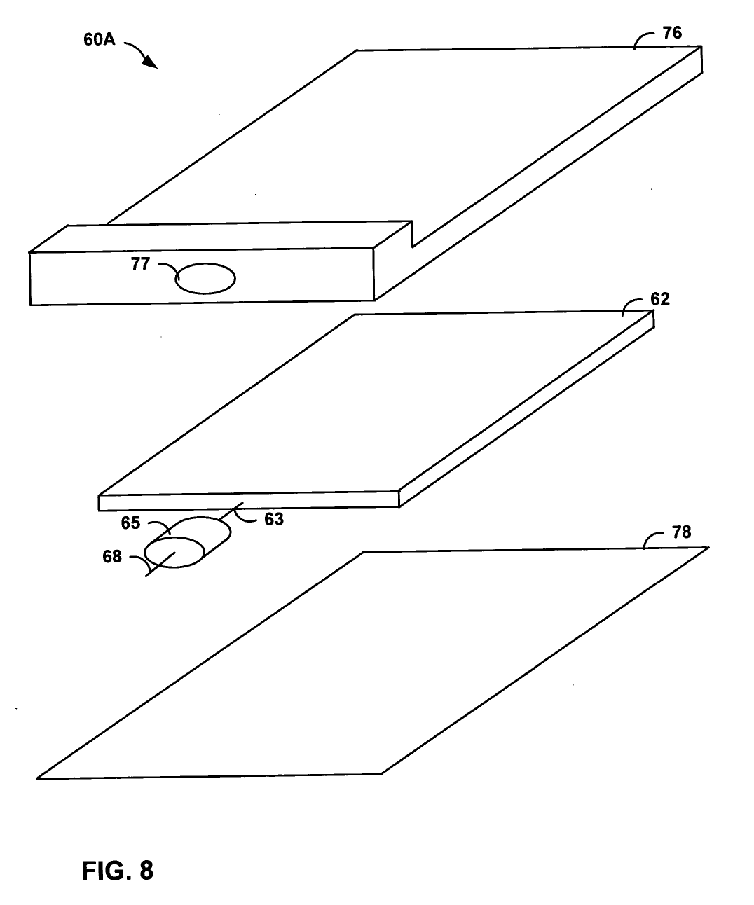

FIG. 8 is an exploded perspective view of battery 60A. As indicated above, battery housings according to the invention may be formed of two or more pieces. In the illustrated embodiment, battery housing 64A of battery 60A is formed from two complementary pieces 76 and 78, which define a cavity to house electrode stack 62. In particular, illustrated housing piece 76 takes the form of a shallow-drawn housing piece that is formed, e.g., pressed, to define the thicknesses T1, T2 of portions P1, P2 of the overall battery housing 64A, while illustrated housing piece 78 takes the form of a substantially flat cover that may be welded to the open “bottom” of piece 76. However, in other embodiments, both battery housing pieces 76, 78 may be formed to define two or more thicknesses of a battery housing 64.

As shown in FIG. 8, an opening 77 may be formed in, e.g., punched through, housing piece 76, through which feedthrough 65 may extend. In some embodiments, the insulative member of feedthrough 65 may be bonded to a ferrule (not shown), which is a part of the battery housing 64A, and may be formed of titanium, stainless steel, or the like. The ferrule may be inserted through opening 77 and welded to housing 64A, either before or after being bonded to feedthrough 65.

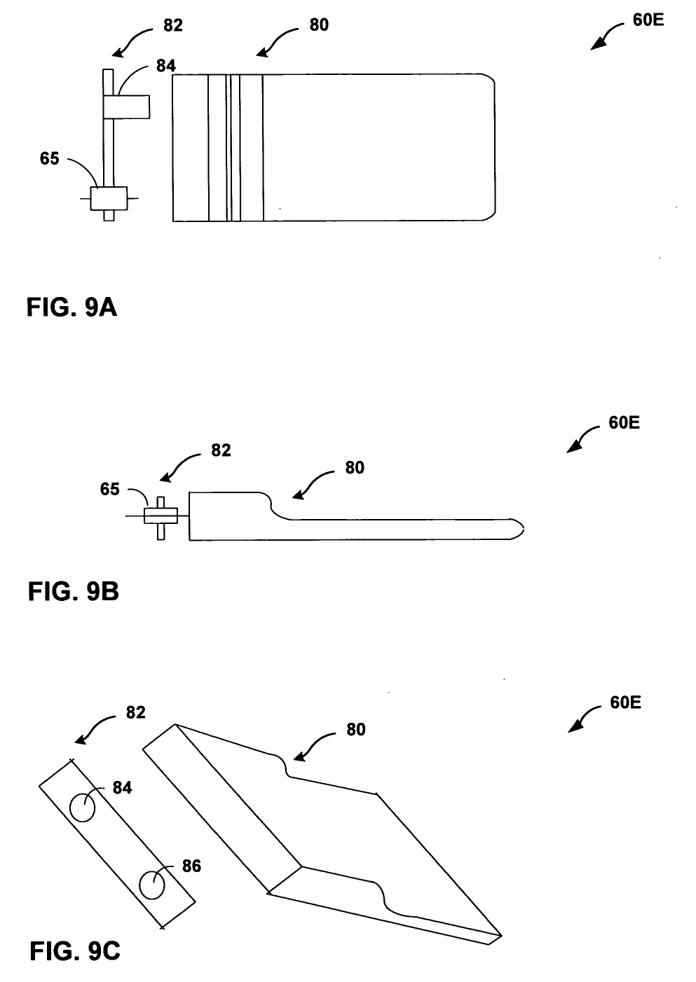

FIGS. 9A-9C are exploded top, side, and perspective views, respectively, that illustrate another example battery 60E according to the invention. In the illustrated embodiment, the housing of battery 60E is formed of two complimentary pieces 80 and 82, which define a cavity to house an electrode stack (not shown). In particular, illustrated housing piece 80 takes the form of a deep-drawn housing piece that is formed, e.g., pressed, to define thicknesses T1, T2 of portions P1, P2 of the overall battery housing, while illustrated housing piece 82 takes the form of a substantially flat cover that may be welded to the open “end” of piece 80.

The housing of battery 60E includes fill port 84, which is an opening that allows battery 60E to be filled with an electrolyte, and is sealed when battery 60E is filled. As shown in FIG. 9, fill port 84 may be formed on piece 82. Piece 82 also includes an opening 86 through which a feedthrough 65 may extend, which may be punched into piece 82 as described above. Feedthrough 65 may be bonded to a ferrule, which is in turn inserted through opening 86 and welded to the housing of battery 60E, as described above. The electrode stack may be coupled to feedthrough 65, and inserted into piece 80 as piece 82 including feedthrough 65 is positioned over the end of piece 80. After an electrode stack is inserted into the cavity defined by piece 80 and piece 82 is welded to piece 80, fill port 84 may be used to fill battery 60E with an electrolyte and subsequently sealed.

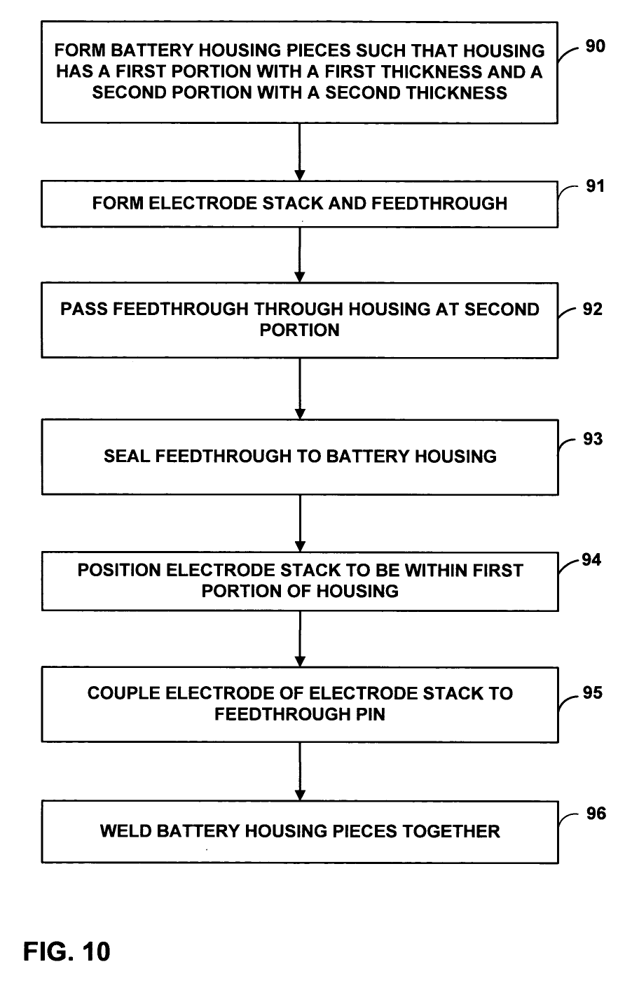

FIG. 10 is a flow diagram illustrating a method of manufacture for a battery 60 according to the invention. Battery housing pieces, such as battery housing pieces 76, 78 from FIG. 8 or battery housing pieces 80, 82 from FIG. 9, are formed for a battery housing 64 such that the battery housing has a first portion P1 with a first thickness T1 and a second portion P1 with a second thickness T2 (90). At least one of the battery housing pieces may be, for example, formed of titanium or stainless steel by pressing, and the battery housing pieces may include a shallow of deep-drawn piece and a cover, as described with reference to FIGS. 8 and 9.

In addition, an electrode stack 62 and a feedthrough 65 are formed (91). As described above, the feedthrough 65 include insulative material, and a feedthrough conductive element 66 passes through and is sealed to the insulative material of the feedthrough 65. The insulative material could be, for example, glass or ceramic material that is melted and bonded, or brazed, to the respective feedthrough conductive element 66. In some embodiments, the feedthrough, e.g., the insulative material of the feedthrough, is bonded or brazed to a metallic ferrule, which forms a part of the battery housing 64, as described above.

Feedthrough 65 is positioned to pass through the second portion P2 of the housing, e.g., through an opening 77, 86 punched through a housing piece 76, 82 (92). Feedthrough 65 is then hermetically sealed to the battery housing 64, e.g., via welding of the ferrule to the housing (93). As described above, feedthrough 65 may be sealed to a ferrule before or after the ferrule is welded to the battery housing 64.

Electrode stack 62 is positioned to be within the first portion P1 of the battery housing 64 (94). At least one of the electrodes of electrode stack 62 is coupled to a feedthrough pin 66 by an interconnect 63, either before or after the electrode stack is positioned within the housing 64, as described above (95). In one embodiment, one of the electrodes is connected to a feedthrough pin 66, and one of the electrodes is connected to the housing 64. The battery housing pieces 76, 78 or 80, 82 are then welded together (96) to hermetically seal the battery 60. As described above, the battery 60 may then be filled with an electrolyte via a fill port 84.

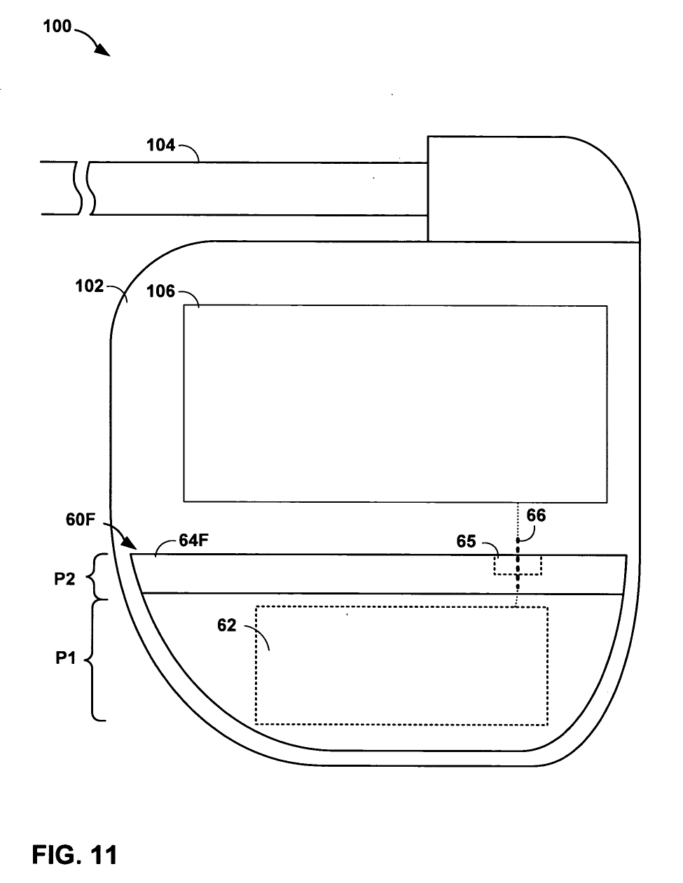

FIG. 11 is a side view of a non-modular IMD 100 that includes a battery 60F with a non-uniform thickness. Battery 60F includes an electrode stack 62 and battery housing 64F. Electrode stack 62 supplies power to components within IMD 100, which may provide stimulation therapy to a patient via lead 104. For example, electrode stack 62 may supply power to a component 106, which may be a circuit board that carries control electronics that control the functioning of IMD 100.

As shown, electrode stack 62 and housing 64F may be contoured to fit within a portion of IMD 100. Further, in the illustrated example, battery housing 64F has at least two thicknesses extending out of the page. In particular, a first portion P1 of battery housing 64F has a first thickness and houses electrode stack 62, and a second portion P2 of battery housing 64F has a second thickness and includes feedthrough 65. With the thickness of portion P1 of the battery housing decreased as allowed by the thickness of electrode stack 62, the thickness of at least a portion of IMD 100 may also be decreased. Alternatively or additionally, a component, such as component 106, may be placed over portion P1 of battery housing 64E. By stacking a component on battery housing 64E, the footprint of IMD 100 may be decreased.

Various embodiments of the invention have been described. Battery housings with at least two thicknesses have been described in the context of an IMD, such as a modular IMD for neurostimulation. Alternatively, battery housings 64 with at least two thicknesses may be used in the context of any IMD, or even in devices other than IMDs that use a battery as a source of power. Battery housings 64 with at least two thicknesses may be used by any device that might benefit from having a more compact battery housing.

Although the shape of battery housings 64 has been exemplified above as prismatic, the prismatic shape is merely exemplary. Alternative embodiments of battery housings 64 may be shaped much differently, while still holding to the principles of the invention. For example, one or more portions of a battery housing 64 may have rounded shapes or edges. In addition, some battery housing embodiments may include a tapered portion between the first and second portions P1, P2 of the battery housing 64 so that the thickness transition between the first and second portion is not so extreme. Alternatively, in some embodiments, the second portion P2 of the housing may consist only of one or more ferrules that extend out from the remainder of the battery housing P1 to a thickness that is greater than the remainder o the battery housing.

In some embodiments, the volume within the second portion P2 of a battery housing 64 may include components in addition to a feedthrough 65. For example, as discussed above, the second portion P2 of a battery housing 64 may include one or more electrical interconnects 63 that couple an electrode of an electrode stack to a feedthrough pin 66. As another example, as discussed above, a second portion P2 may include a fill port 84. Other examples of components that may be included with a second portion P2 of a battery housing 64 include a reference electrode or sensor for battery diagnosis, a fuse, an electronic component, or an insulator.

Although described herein as defined by the feedthrough 65, in some embodiments, the thickness T2 of a second portion P2 of a battery housing 64 is defined by one or more of these additional components. A fill port 84, in particular, may in some embodiments be larger than a feedthrough 82, and may define the thickness T2 of a second portion P2 of a battery housing 64. These and other embodiments are within the scope of the following claims.

1. A battery comprising:
an electrode stack;
a feedthrough coupled to the electrode stack; and
a battery housing including a first portion that houses the electrode stack and a second portion that includes the feedthrough, wherein a thickness of the second portion is greater than a thickness of the first portion.

2. The battery of claim 1, wherein the thickness of the first portion of the battery housing is defined by the electrode stack, and the thickness of the second portion of the battery housing is defined by the feedthrough.

3. The battery of claim 1, wherein the thickness of the first portion of the battery housing is within a first range from approximately 1 mm to approximately 5 mm, and the thickness of the second portion of the battery housing is within a second range from approximately 3 mm to approximately 10 mm.

4. The battery of claim 1, wherein the thickness of the first portion of the battery housing is approximately 3 mm, and the thickness of the second portion of the battery housing is approximately 6 mm.

5. The battery of claim 1, wherein the thickness of the first portion of the battery housing is less than approximately 80 percent of the thickness of the second portion of the battery housing.

6. The battery of claim 1, wherein the thickness of first portion of the battery housing is approximately 60 percent of the thickness of the second portion of the battery housing.

7. The battery of claim 1, wherein a width of the second portion of the battery housing is less than a width of the first portion of the battery housing.

8. The battery of claim 1, wherein the battery housing is substantially prismatic.

9. The battery of claim 1, wherein the battery housing is curved along at least one axis.

10. The battery of claim 1, wherein the battery housing is hermetic and is hermetically sealed to the feedthrough.

11. The battery of claim 1, wherein the feedthrough comprises a conductor that is coupled to the electrode stack, passes through an insulative member, and is sealed to the insulative member.

12. The battery of claim 11, wherein the conductor comprises a conductive pin and the insulative member is formed of glass or ceramic.

13. The battery of claim 1, wherein the second portion of the battery housing includes a plurality of feedthroughs.

14. The battery of claim 1, wherein the second portion of the battery housing includes a fill port.

15. The battery of claim 1, wherein the second portion of the battery housing houses at least one of an interconnect that connects the feedthrough to the electrode stack, a reference electrode, a sensor, a fuse, an electronic component, or an insulator.

16. The battery of claim 1, wherein the feedthrough extends through the battery housing in a direction perpendicular to a long axis of the battery.

17. A battery comprising:
an electrode stack;
a feedthrough coupled to the electrode stack; and
a battery housing that houses the electrode stack and includes the feedthrough, wherein the battery housing includes a first portion with a thickness defined by the electrode stack, and a second portion with a thickness defined by the feedthrough.

18. The battery of claim 17, wherein the thickness of the second portion of the battery housing is greater than the thickness of the first portion of the battery housing.

19. The battery of claim 17, wherein the thickness of the first portion of the battery housing is within a first range from approximately 1 mm to approximately 5 mm, and the thickness of the second portion of the battery housing is within a second range from approximately 3 mm to approximately 10 mm.

20. The battery of claim 17, wherein the thickness of the first portion of the battery housing is approximately 3 mm, and the thickness of the second portion of the battery housing is approximately 6 mm.

21. The battery of claim 17, wherein the thickness of the first portion of the battery housing is less than approximately 80 percent of the thickness of the second portion of the battery housing.

22. The battery of claim 17, wherein the thickness of first portion of the battery housing is approximately 60 percent of the thickness of the second portion of the battery housing.

23. The battery of claim 17, wherein a width of the second portion of the battery housing is less than a width of the first portion of the battery housing.

24. The battery of claim 17, wherein the battery housing is substantially prismatic.

25. The battery of claim 17, wherein the battery housing is curved along at least one axis.

26. The battery of claim 17, wherein the battery housing is hermetic and is hermetically sealed to the feedthrough.

27. The battery of claim 17, wherein the feedthrough comprises a conductor that is coupled to the electrode stack, passes through an insulative member, and is sealed to the insulative member.

28. The battery of claim 27, wherein the conductor comprises a conductive pin and the insulative member is formed of glass or ceramic.

29. The battery of claim 17, wherein the second portion of the battery housing includes a plurality of feedthroughs.

30. The battery of claim 17, wherein the second portion of the battery housing includes a fill port.

31. The battery of claim 17, wherein the second portion of the battery housing houses at least one of an interconnect that connects the feedthrough to the electrode stack, a reference electrode, a sensor, a fuse, an electronic component, or an insulator.

32. The battery of claim 17, wherein the feedthrough extends out of the battery housing in a direction perpendicular to a long axis of the battery housing.

33. An implantable medical device comprising:
a housing; and
a battery located within the housing comprising:
an electrode stack to provide power for the implantable medical device;
a feedthrough coupled to the electrode stack; and
a battery housing including a first portion that houses the electrode stack and a second portion that includes the feedthrough, wherein a thickness of the second portion is greater than a thickness of the first portion.

34. The implantable medical device of claim 33, wherein the thickness of the first portion of the battery housing is within a first range from approximately 1 mm to approximately 5 mm, and the thickness of the second portion of the battery housing is within a second range from approximately 3 mm to approximately 10 mm.

35. The implantable medical device of claim 33, wherein the thickness of the first portion of the battery housing is less than approximately 80 percent of the thickness of the second portion of the battery housing.

36. The implantable medical device of claim 33, wherein the second portion of the battery housing includes a plurality of feedthroughs.

37. The implantable medical device of claim 33, wherein the second portion of the battery housing includes a fill port.

38. The implantable medical device of claim 33, further comprising an implantable medical device component that is located over the first portion of the battery housing and substantially adjacent to the second portion of the battery housing.

39. The implantable medical device of claim 38, wherein the component comprises a circuit board.

40. The implantable medical device of claim 38, wherein the component comprises a secondary coil.

41. A modular implantable medical device comprising a plurality of interconnected modules, wherein one of the modules comprises a battery, the battery comprising:
an electrode stack to provide power for the modular implantable medical device;
a feedthrough coupled to the electrode stack; and
a battery housing including a first portion that houses the electrode stack and a second portion that includes the feedthrough, wherein a thickness of the second portion is greater than a thickness of the first portion.

42. The modular implantable medical device of claim 41, wherein the thickness of the first portion of the battery housing is within a first range from approximately 1 mm to approximately 5 mm, and the thickness of the second portion of the battery housing is within a second range from approximately 3 mm to approximately 10 mm.

43. The modular implantable medical device of claim 41, wherein the thickness of the first portion of the battery housing is less than approximately 80 percent of the thickness of the second portion of the battery housing.

44. The modular implantable medical device of claim 41, wherein the second portion of the battery housing includes a plurality of feedthroughs.

45. The modular implantable medical device of claim 41, wherein the second portion of the battery housing includes a fill port.

46. The modular implantable medical device of claim 41, wherein another of the plurality of modules is located over the first portion of the battery housing of the battery housing and substantially adjacent to the second portion of the battery housing.

47. The modular implantable medical device of claim 46, wherein the other module is a control module that includes control electronics.

48. The modular implantable medical device of claim 46, wherein the other module is a recharge module that includes a secondary coil.

49. The modular implantable medical device of claim 41, wherein the battery housing is curved along at least one axis.

50. The modular implantable medical device of claim 41, wherein the modular implantable medical device comprises at least one of an implantable neurostimulator or an implantable pump.

51. The modular implantable medical device of claim 41, wherein the modular implantable medical device is configured for implantation on a cranium of a patient.

52. The modular implantable medical device of claim 41, further comprising a member that at least partially encapsulates the plurality of modules.

53. A method of making a battery that comprises an electrode stack, a feedthrough coupled to the electrode stack, and a battery housing, the method comprising:
forming at least one of a plurality of pieces of the housing such that a thickness of a first portion of the battery housing is less than a thickness of a second portion of the battery housing;
positioning the electrode stack within the first portion of the battery housing; and
positioning the feedthrough to pass through the battery housing at the second portion of the battery housing.

54. The method of claim 53, wherein forming at least one of a plurality of pieces of the housing comprises pressing the at least one of the plurality of pieces such that a thickness of a first portion of the battery housing is less than a thickness of a second portion of the battery housing.

55. The method of claim 53, wherein forming at least one of a plurality of pieces of the housing comprises forming one of a shallow-drawn piece and a deep-drawn piece such that a thickness of a first portion of the battery housing is less than a thickness of a second portion of the battery housing.

56. A battery comprising:
an electrode stack;
a fill port; and
a battery housing that houses the electrode stack and includes the fill port, wherein the battery housing includes a first portion with a thickness defined by the electrode stack, and a second portion with a thickness defined by the fill port.

57. The battery of claim 56, wherein a thickness of the second portion of the battery housing is greater than a thickness of the first portion of the battery housing.
